(19) United States
(12) Patent Application Publication
(10) Pub. No.: US 2021/0401918 A1
(43) Pub. Date: Dec. 30, 2021

(54) COMPOSITION COMPRISING COMBINATION OF RED CLOVER EXTRACT AND HOPS EXTRACT FOR IMPROVEMENT OF MENOPAUSAL DISORDER

(71) Applicant: NATURETECH CO. LTD., Chungcheongbuk-do (KR)

(72) Inventors: Young Chul LEE, Daejeon (KR); Su Yeol YU, Incheon (KR); Su Hyun YU, Seoul (KR); Hyun Jin KIM, Chungcheongnam-do (KR); Se Yeong JEON, Chungcheongbuk-do (KR); Bo Su LEE, Incheon (KR); Mi Ran KIM, Daejeon (KR); Jeong Jun LEE, Gyeonggi-do (KR)

(21) Appl. No.: 17/290,058

(22) PCT Filed: Nov. 12, 2019
(86) PCT No.: PCT/KR2019/016043

§ 371 Date: Apr. 29, 2021

(30) Foreign Application Priority Data
Nov. 23, 2018 (KR) 10-2018-0146065

(51) Int. Cl. A61K 36/48; A61K 36/185; A61P 5/30; A23L 33/105

(52) U.S. Cl. CPC A61K 36/48; A61K 36/185; A61P 5/30; A23V 2002/00; A61K 2236/15; A61K 2236/53; A61K 2236/51; A23L 33/105

(57) ABSTRACT

The present invention relates to a composition for improvement of female menopausal (climacteric) disorders, comprising as an active ingredient a combination of a red clover extract and a hops extract, more specifically a food composition for improvement of women's menopausal disorders or a pharmaceutical composition for prevention or treatment of women's menopausal disorders, comprising as an active ingredient a combination of a red clover extract and a hops extract; and a method for preparing said composition for improvement of women's menopausal disorders.

### TECHNICAL FIELD

The present invention relates to a composition for improvement of women's menopausal (climacteric) disorders, comprising as an active ingredient a combination of a red clover extract and a hops extract, more specifically a food composition for improvement of women's menopausal disorders or a pharmaceutical composition for prevention or treatment of women's menopausal disorders, comprising as an active ingredient a combination of a red clover extract and a hops extract; and a method for preparing said composition for improvement of women's menopausal disorders.

This research was financially supported by the Ministry of SMEs and Startups (MSS), Korea, under the “Regional Specialized Industry Development Program (R&D, P0003019)” supervised by the Korea Institute for Advancement of Technology (KIAT).

### BACKGROUND ART

Menopause is the disruption of menstruation, which occurs when the genetically determined ovary function of about 50 years after a woman's birth reaches the end of its lifespan. Menopause means loss of fertility and is a physiological change rather than a pathological phenomenon. Currently, the life expectancy of Korean women is 85.4 years (2016, Statistics Korea). Assuming that the average menopausal age of Korean women is 50 years as defined by the Korean Academy of Obstetrics and Gynecology, it means that more than one-third of a woman's lifetime is depleted of female hormones.

During menopause, changes occur throughout the body including the vascular system, musculoskeletal system, genitourinary system and cranial nerves, due to secretion imbalances and reductions in female hormones. In other words, menopause is accompanied by various diseases including vasomotor symptoms and psychological symptoms such as hot flashes, night sweats, sleep disorders, fatigue, depression, anxiety, concentration disorders and memory impairment; dyspareunia and frequent urination due to urogenital atrophy; loss of skin elasticity and breast sagging due to decreased collagen; cardiovascular and musculoskeletal symptoms; and dementia. Although menopausal (climacteric) symptoms vary from person to person, it has been reported that the more menopausal symptoms, the more severe the disease, and the longer the duration, the worse the quality of life of women. In addition, menopausal symptoms are more likely to develop into chronic diseases with physical aging.

To improve these menopausal disorders, hormonal therapies have been used to replenish scarce female hormones, but long-term use has resulted in uterine cancer, breast cancer, stroke, pulmonary thromboembolism, and various side effects. Hence, recently, plant-derived phytoestrogen has attracted attention as an alternative. It is known that the phytoestrogens are structurally similar to the estrogens and bind to the estrogen receptors ER-alpha and ER-beta to show a weak estrogen-like effect, thereby suppressing and ameliorating various metabolic syndromes according to the menopause of women, and exhibiting relatively good bone mass preservation effect. Licorice, hops, pomegranate and red clover are known as plants containing such phytoestrogens. In this regard, Korean Patent Laid-Open Publication No. 2016-0011756 A discloses a functional food composition for preventing and improving female climacteric or menopausal symptoms, comprising soy isoflavone extract, hops extract and licorice extract. In addition, studies on improving menopausal disorders using various phytoestrogen-containing plants are being conducted.

### PRIOR ART DOCUMENTS

S. R. Milligan, et al. (1999), Identification of a potent phytoestrogen in hops (Humulus lupulus L.) and beer, The Journal of Clinical Endocrinology & Metabolism, Vol. 83, No. 6, pp. 2249-2252.
E. Dornstauder, et al. (2001), Estrogenic activity of two standardized red clover extracts (Menoflavon®) intended for large scale use in hormone replacement therapy, Journal of steroid biochemistry & molecular biology, Vol. 78, pp. 67-75.
Atieh Hajirahimkhan, et al. (2013), Evaluation of Estrogenic Activity of Licorice Species in Comparison with Hops Used in Botanicals for Menopausal Symptoms, PLOS ONE, Vol. 8, Issue 7.
Joanna E. Burdette, et al. (2002), Trifolium pretense (Red Clover) Exhibits Estrogenic Effects In Vivo in Ovariectomized Sprague-Dawley Rats, American society for nutritional sciences, Biochemical and Molecular Action of Nutrients Research Communication.
Cassia R. Overk emd (2005), Comparison of the In Vitro Estrogenic Activities of Compounds from Hops (Humulus lupulus) and Red Clover (Trifolium pratense), J Agric Food Chem., 53(16), pp. 6246-6253.
Michael Humpel, et al. (2005), Tissue specificity of 8-prenylnaringenin: Protection from ovariectomy induced bone loss with minimal tropic effects on the uterus, Journal of steroid biochemistry & molecular biology, Vol. 97, pp. 299-305.
Paola Spagnuolo, et al. (2014), Isoflavone content and estrogenic activity of different batches of red clover (Trifolium pratense L.) extracts: An in vitro study in MCF-7 cells, Fitoterapia, Vol. 94, pp. 62-69.
James Bowe, et al. (2006), The hop phytoestrogen, 8-prenylnaringenin, reverses the ovariectomy-induced rise in skin temperature in an animal model of menopausal hot flushes, Journal of Endocrinology, Vol. 191, pp. 399-405.
Shu-Jem Su, et al. (2013), The Preventive Effect of Biochanin A on Bone Loss in Ovariectomized Rats: Involvement in Regulation of Growth and Activity of Osteoblasts and Osteoclasts, Evidence-Based Complementary and Alternative Medicine.
Evelyne Reiter, et al. (2011), Red clover and soy isoflavones an in vitro safety assessment, Gynecological endocrinology, Vol. 27, Issue 12, pp. 1037-1042.

### DISCLOSURE OF INVENTION

### Technical Problem

The present invention is intended to provide a combination of a red clover extract and a hops extract, thereby to provide a composition that can improve menopausal disorders and menopausal symptoms of women without side effects and more effectively compared to when the two extracts are applied individually.

However, the problem to be solved by the present invention is not limited to the above-mentioned problem, and other problems that are not mentioned will be clearly understood by those skilled in the art from the following description.

### Solution to Problem

The first aspect of the present invention relates to a composition for improvement of women's menopausal disorders, comprising as an active ingredient a combination of a red clover extract and a hops extract.

The second aspect of the present invention relates to a food composition for improvement of women's menopausal disorders, comprising the composition according to the first aspect.

The third aspect of the present invention relates to a pharmaceutical composition for prevention or treatment of women's menopausal disorders, comprising the composition according to the first aspect.

The fourth aspect of the present invention relates to a method for preparing a composition for improvement of women's menopausal disorders, comprising the following steps: (a) pulverizing a red clover leaf and then extracting it in alcohol to prepare a red clover extract; (b) extracting hops flowers and hops buds in alcohol to prepare a hops extract; and (c) mixing the red clover extract and the hops extract.

The fifth aspect of the present invention relates to a combination of a red clover extract and a hops extract for use in the improvement, prevention or treatment of women's menopausal disorders.

The sixth aspect of the present invention relates to a method of improving, preventing or treating women's menopausal disorders in a subject, comprising administering to the subject an effective amount of a combination of a red clover extract and a hops extract.

The above-mentioned solutions to the problems are merely exemplary and should not be construed to limit the present invention. Besides the exemplary embodiments described above, there may be additional embodiments and examples described in the drawings and detailed description of the invention.

### Advantageous Effects of Invention

The composition of the present invention comprising a red clover extract and a hops extract shows a synergistic effect (synergism) greater than the expected effect when the two extracts are applied individually, in the improvement of women's menopausal disorders, thereby being able to effectively improve representative typical women's menopausal disorders such as blood circulation disorder, weight gain, hot flashes, hyperlipidemia and osteoporosis.

### BRIEF DESCRIPTION OF DRAWINGS

FIG. 1 shows a high performance liquid chromatography (HPLC) result of analyzing the contents of Daidzein, Genistein, Formononetin and Biochanin A in the red clover extract.

FIG. 2 shows an HPLC result of analyzing the contents of Isoxanthohumal, 8-Prenylnaringenin and Xanthohumol in the hops extract.

FIG. 3 shows an HPLC result of analyzing the contents of Biochanin A and Xanthohumol in the combination of the red clover extract and the hops extract.

### BEST MODE FOR CARRYING OUT THE INVENTION

Hereinafter, embodiments of the present invention will be described in detail so that those skilled in the art can easily carry out the present invention. However, the present invention can be implemented in many different forms and will not be limited to the embodiments illustrated herein.

Throughout this specification, when a part is said to “comprise” a certain component, it does not mean that said part excludes other components but means that said part can further include the other components, unless stated otherwise.

Throughout this specification, the term “improvement” or “treatment” means any action that mitigates or beneficially alters women's menopausal disorders by administration of the composition, and the term “prevention” means any action that suppresses or delays onset of women's menopausal disorders by administration of the composition.

Hereinafter, the composition for improvement of women's menopausal disorders, the food composition and the pharmaceutical composition comprising the same, and the method for preparing the same of the present invention will be described in detail with reference to embodiments, examples and drawings. However, the present invention is not limited to these embodiments, examples and drawings.

The first aspect of the present invention provides a composition for improvement of women's menopausal disorders, comprising as an active ingredient a combination of a red clover extract and a hops extract.

Red clover (Trifolium pretense L) is a perennial plant of the legume family and is also called Black Tea Chukcho, red rabbit herb, red ginseng leaf or gold coin herb. It is native to the Mediterranean coast and Southwest Asia, and is an upright herb that is widely distributed and cultivated around the world. Red clover is also used for feeding, and in spring, the young leaves are slightly boiled to be eaten as herbs. Red clover may be divided into aerial parts and root parts. As the extract of the present invention, the aerial parts of red clover, for example, a leaf extract, may preferably be used, but the present invention is not limited thereto.

Hops (Homulus lupulus L) is a vine plant native to Europe, Australia and North America. Many compounds present in the hops extracts exhibit various physiological activities such as anti-cancer, osteoporosis improvement, anti-oxidation and whitening, and among them, 8-prenylnaringenin, isoxanthohumol and the like are known to have estrogen activity. As the extract of the present invention, an extract obtained from a flower or a bud may be used, but the present invention is not limited thereto.

According to one embodiment of the present invention, the composition may comprise a red clover extract and a hops extract in a weight ratio of 10:1 to 1:5, and for example, the weight ratio may be 10:1 to 1:2, 10:1 to 1:1, 5:1 to 1:5, 5:1 to 1:1, 5:1 to 2:1, 3:1 to 1:5, 3:1 to 1:1, 3:1 to 2:1, 5:1, 4:1, 3:1, 2:1, 1:1, 1:2 or 1:3, but may not be limited thereto.

According to one embodiment of the present invention, the menopausal disorders may be one or more menopausal symptoms selected from the group consisting of blood circulation disorders, weight gain, hot flashes, osteoporosis, blood cholesterol increase, sweating, insomnia, nervousness, depression, dizziness, lack of concentration, joint pain, headache, palpitations, vagina dryness, fatigue, excitement, sleep disorders, memory loss, memory disorders, forgetfulness, cold sweats, pounding, frequent urination, anxiety and atherosclerosis, but may not be limited thereto. The menopausal disorders may include any menopausal symptoms caused directly or indirectly by female hormone deficiency.

According to one embodiment of the present invention, the red clover extract may be extracted from the red clover leaves, but may not be limited thereto.

According to one embodiment of the present invention, the hops extract may be extracted from hops flowers, hops buds, or both, but may not be limited thereto.

According to one embodiment of the present invention, the red clover extract and the hops extract may be red clover extract powder and hops extract powder, respectively, but may not be limited thereto.

The second aspect of the present invention provides a food composition for improvement of women's menopausal disorders, comprising the composition according to the first aspect.

Detailed descriptions are omitted for the parts overlapping with the first aspect of the present invention, but the descriptions illustrated for the first aspect of the present invention may be equally applied to the second aspect of the present invention even if they are omitted for the second aspect of the present invention.

Food compositions that may comprise a combination of a red clover extract and a hops extract include, for example, various foods, powders, granules, tablets, capsules, syrups, beverages, gums, teas, vitamin complexes, health functional foods and the like, but they are not limited thereto. The combination may also be added to food or beverages.

The amount of the combination in the food or beverage may be about 0.01 to 30% by weight of the total food weight, and in the case of a beverage composition, the amount of the combination may be a ratio of about 0.01 to 30 g based on 100 ml, but may not be limited thereto.

When the food composition of the present invention is a beverage composition, except for containing the above combination of the extracts as an essential ingredient in the above-indicated ratios, there is no particular limitation on other components. Like general beverages, it may contain various flavors or natural carbohydrates as additional ingredients. Examples of the above-mentioned natural carbohydrates include monosaccharides such as glucose and fructose; disaccharides such as maltose and sucrose; and polysaccharides such as conventional sugars including dextrin and cyclodextrin, and sugar alcohols including xylitol, sorbitol and erithritol. As flavors other than those described above, natural flavors (tauumatin, stevia extract (e.g., Rebaudioside A, glycyrrhizin, etc.)) and synthetic flavors (saccharin, aspartame, etc.) may be used, but are not limited thereto. The ratio of the natural carbohydrate may generally be in the range of about 1 to 20 g per 100 ml of the beverage composition comprising the combination of the extracts of the present invention, but may not be limited thereto.

The composition comprising the combination of a red clover extract and a hops extract may contain a variety of nutrients, vitamins, minerals (electrolytes), fragrances including synthetic fragrances and natural fragrances, colorants, pectic acid and salts thereof, alginic acid and salts thereof, organic acids, protective colloidal thickeners, pH adjusters, stabilizers, preservatives, glycerin, alcohols, carbonating agents used in carbonated drinks and the like, but are not limited thereto.

The third aspect of the present invention provides a pharmaceutical composition for prevention or treatment of women's menopausal disorders, comprising the composition according to the first aspect.

Detailed descriptions are omitted for the parts overlapping with the first aspect of the present invention, but the descriptions illustrated for the first aspect of the present invention may be equally applied to the third aspect of the present invention even if they are omitted for the third aspect of the present invention.

The pharmaceutical composition according to the present invention may comprise a pharmaceutically effective amount of the combination of a red clover extract and a hops extract alone, or may further comprise one or more pharmaceutically acceptable carriers, excipients or diluents. The term “pharmaceutically acceptable” refers to a nontoxic composition that is physiologically acceptable, and when administered to humans, does not inhibit the action of the active ingredient, and generally does not cause gastrointestinal disorders, an allergic reaction such as dizziness, or any similar reactions. The “pharmaceutically effective amount” refers to an amount that exhibits a higher response than a negative control, and preferably an amount sufficient to exhibit an effect of preventing and/or treating a menopausal disorder.

As used herein, the term “carrier” can be defined as a compound that facilitates the addition of the active compound into cells or tissues, and may be selected and used without limitation from commonly used carriers that facilitate the introduction of many organic compounds into cells or tissues of the organism.

As used herein, the term “excipient” can be defined as

As used herein, the term “diluents” can be defined as a compound that not only stabilizes the biologically active form of the active compound but also dilutes the active compound in water for dissolution. Salts dissolved in buffer solutions are used as diluents in the art, and the commonly used buffer solutions are phosphate buffered saline, which mimics the salt state of human body fluid, but may not be limited thereto. Since buffer salts at low concentrations control the pH of the solution, the buffer diluents may not modify the biological activity of the active compound.

The carriers, excipients and diluents may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxyl benzoate, propyl hydroxyl benzoate, talc, magnesium stearate and mineral oil. Formulations may be prepared using diluents or excipients such as fillers, extenders, binders, wetting agents, disintegrants, surfactants, etc. which are commonly used.

The pharmaceutical compositions according to the present invention can be used in the form of oral formulations such as tablets, pills, powders, granules, capsules or drinks; external preparations; suppositories; and sterile injectable solutions, each formulated according to conventional methods.

According to one embodiment of the present invention, the pharmaceutical composition may be for oral administration, intraperitoneal administration, intravenous administration, intramuscular administration, subcutaneous administration, intradermal administration, intranasal administration, pulmonary administration or rectal administration, but may not be limited thereto.

Solid formulations for oral administration include tablets, pills, powders, granules, capsules and the like, and such solid formulations may be prepared by mixing the above extracts with at least one excipient such as starch, calcium carbonate, sucrose, lactose or gelatin. In addition to simple excipients, lubricants such as magnesium stearate and talc may also be used. Liquid formulations for oral administration include suspensions, oral solutions, emulsions, syrups and the like, and may comprise water which is a commonly used simple diluent; liquid paraffin; and various excipients, for example, wetting agents, sweeteners, fragrances and preservatives. Formulations for parenteral administration include sterile aqueous solutions, non-aqueous solutions, suspensions, emulsions, lyophilized preparations and suppositories. For the non-aqueous solutions and suspensions, propylene glycol, polyethylene glycol, vegetable oils such as olive oil, injectable esters such as ethyl oleate and the like can be used. For the base of the suppositories, witepsol, macrogol, tween 61, cacao butter, laurin butter, glycerogelatin and the like can be used.

According to one embodiment of the present invention, the pharmaceutical composition may be formulated into tablets, pills, powders, granules, capsules or drinks for oral administration, but may not be limited thereto.

Preferred dosages of the pharmaceutical compositions of the present invention vary depending on the condition and weight of the patient, the degree of the disease, the form of the drug, the route of administration and the duration, and may be appropriately selected by those skilled in the art.

For example, the pharmaceutical composition may be administered in the dosage of about 20 to 1,000 mg/kg/day, about 50 to 1,000 mg/kg/day, about 100 to 1,000 mg/kg/day, about 300 to 1,000 mg/kg/day, about 500 to 1,000 mg/kg/day, about 800 to 1,000 mg/kg/day, about 20 to 100 mg/kg/day, about 20 to 200 mg/kg/day, about 20 to 500 mg/kg/day, about 20 to 800 mg/kg/day, about 50 to 500 mg/kg/day, about 100 to 500 mg/kg/day, about 200 to 500 mg/kg/day, or about 250 to 500 mg/kg/day, but not be limited thereto. The pharmaceutical composition may be administered once a day or may be divided into several portions and administered several times. The above dosage does not limit the scope of the present invention in any aspect.

The fourth aspect of the present invention provides a method for preparing a composition for improvement of women's menopausal disorders, comprising the following steps: (a) pulverizing a red clover leaf and then extracting it in alcohol to prepare a red clover extract; (b) extracting hops flowers and hops buds in alcohol to prepare a hops extract; and (c) mixing the red clover extract and the hops extract.

For example, the alcohol extraction may be performed using about 50% to 90% alcohol, or about 60% to 70% alcohol, and about 60% alcohol may be preferable for the extraction of red clover, and about 70% alcohol may be preferable for the extraction of hops.

For example, the extraction may be performed at about 50 to 90° C., about 60 to 80° C., or about 70° C., and the extraction is performed at least once, for example, twice, three times, four times or more. The extraction time may be about 1 hour to 10 hours, about 2 hours to 6 hours, or about 4 hours.

According to one embodiment of the present invention, the steps (a) and (b) may further include additional steps of filtering, concentrating and drying the extract after alcohol extraction, but may not be limited thereto.

The filteration step can be performed by any filtration methods known in the art without limitation, and for example, it can be carried out using a filter paper. The concentration step also can be performed by any concentration methods known in the art without limitation, and for example, a vacuum concentration can be used.

When the extract is dried, an extract powder can be finally prepared.

The fifth aspect of the present invention provides a combination of a red clover extract and a hops extract for use in the improvement, prevention or treatment of women's menopausal disorders.

The sixth aspect of the present invention provides a method of improving, preventing or treating women's menopausal disorders in a subject, comprising administering to the subject an effective amount of a combination of a red clover extract and a hops extract.

The detailed descriptions illustrated for the first to third aspects of the present invention may be equally applied to the fifth and sixth aspects of the present invention even if they are omitted herein.

Hereinafter, the present invention will be described in more detail with reference to the following examples, but the following examples are provided for illustrative purposes only and are not intended to limit the scope of the present invention.

### EXAMPLES

### Preparation Example 1: Preparation of Red Clover Extract

The red clover leaf was pulverized by a grinder, and then extracted in reflux twice at 70° C. for 4 hours using 10 L of 60% alcohol (Daehan Alcohol Life Co., Ltd., Korea) for 1 kg of the raw material. The extract was filtered under reduced pressure through a filter paper (ADVANTEC 2, 280 mm, China), the filtrate was concentrated in vacuo (rotavaper R-220SE, BUCHI, Switzerland) and dried in a dryer (Daeil Engineering, Korea) to obtain an extract in powder form.

### Preparation Example 2: Preparation of Hops Extract

1 kg of well-dried hops flowers and buds were extracted in reflux twice at 70° C. for 4 hours using 10-L of 70% alcohol. The extract was filtered through a filter paper, the filtrate was concentrated in vacuo and dried in a dryer to obtain an extract in powder form.

### Preparation Example 3: Preparation of Combination of Red Clover Extract and Hops Extract

The red clover extract and the hops extract obtained in Preparation Example 1 and Preparation Example 2, respectively, were mixed in a weight ratio of 2:1 or 3:1 to prepare a combination of red clover extract and hops extract. These combinations were used in the evaluation of efficacy in the in vitro test and in vivo test described below.

### Experimental Example 1: HPLC Analysis of Red Clover Extract

The content of major components in the red clover extract was measured using an Agilent HPLC 1200 series/DAD detector (Aligent technologies, USA). Formononetin (Wuhan Chemfaces Biochemical Co., Ltd. China), biochanin A (Sigma-aldrich, USA), genistein (Wuhan Chemfaces Biochemical Co., Ltd. China) and daidzein (Wuhan Chemfaces Biochemical Co., Ltd. China) are representative isoflavones contained in red clover. Hence, quantitative analysis for the four isoflavones was perfomed.

A C18 4.5×250 mm, 5 μm column (Phenomenex, USA), C-18 Security Guard Cartridge 4.0×3.0 mm (Phenomenex, USA), Security Guard Cartridge Holder (Phenomenex, USA) were used for the analysis. (A) Acetonitrile (ACN, for HPLC use, J. T. Baker, USA) and (B) distilled water (aquaMAX™-ultra, YoungRin Machinery, Korea) containing 0.1% phosphoric acid (Sigma-Aldrich, USA) were used as mobile phase. The gradient conditions of the solvents are shown in Table 1 below. The solvents were flowed at a rate of 0.8 ml per minute at a column temperature of 25° C., 10 μl was injected, and absorbance was measured at 260 nm.

TABLE 1

| Time (min) | A % | B % |
|---|---|---|
| 0 | 25 | 75 |
| 30 | 70 | 30 |
| 32 | 25 | 75 |
| 40 | 25 | 75 |

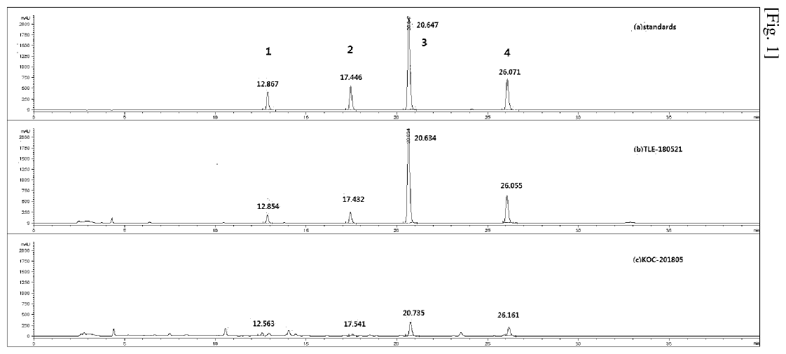

The standard product was extracted by ultrasonic (power sonic 520, Korea) for 10 minutes using 100% methanol (MeOH, for HPLC use, J. T. Baker, USA) at a concentration of 1 mg/l ml. Each standard product was aliquoted and stored in a −20° C. freezer, and used in dilution for analysis. The extract sample was placed in a 50 ml volumetric flask to a concentration of about 200 mg/50 ml, and sonicated for 20 minutes by adding 40 ml of 100% methanol. When coupled to room temperature, the flask was added with 100% methanol to the indicated line and mixed well. Then, the extract sample was filtered through 0.45 μm PTFE filter (Whatman, UK) and analyzed. HPLC analysis results of the major components of the regional and seasonal red clover extracts are shown in Table 2 below and FIG. 1 (1: daidzein, 2: genistein, 3: formononetin, 4: biochanin A).

TABLE 2

| Code | Daidzein | Genistein | Formononetin | Biocahnin A | Total |
|---|---|---|---|---|---|
| RCE-171015** | 0.43 | 0.57 | 17.84 | 2.18 | 21.02 |
| RCE-180521** | 1.00 | 1.00 | 5.95 | 1.61 | 9.56 |
| TLE-180521** | 0.51 | 0.52 | 7.58 | 1.69 | 10.3 |
| RCE-180526* | 0.07 | 0.02 | 0.39 | 0.08 | 0.56 |
| KOC-2180326-1* | 0.19 | 0.11 | 1.52 | 0.62 | 2.44 |
| KOC-20180326-2* | 0.06 | 0.02 | 1.03 | 0.16 | 1.27 |
| KOC-2180326-4* | 0.09 | 0.04 | 1.12 | 0.29 | 1.54 |
| KOC-40E* | 0.16 | 0.08 | 0.38 | 0.62 | 1.24 |
| KOC-80E* | 0.28 | 0.15 | 1.96 | 0.9 | 3.29 |
| KOC-201805* | 0.23 | 0.09 | 1.08 | 0.57 | 1.97 |
| RCE-0704* | 0.14 | 0.06 | 0.82 | 0.36 | 1.38 |
| RCE-0808* | 0.13 | 0.04 | 0.71 | 0.29 | 1.17 |
| RC-Leaf* | 0.19 | 0.11 | 1.52 | 0.62 | 2.44 |

*red clover extract samples,
**commercial product samples

### Experimental Example 2: HPLC Analysis of Hops Extract

The content of three major components in the hops extract was measured using an Agilent HPLC/PDA system. Isoxanthohumol (IXN) (Wuhan Chemfaces Biochemical Co., Ltd. China), 8-prenylnaringenin (8-PN) (Wuhan Chemfaces Biochemical Co., Ltd. China) and xanthohumol (Sigma-Aldrich, USA) are representative components of hops. Luna C18 4.5×250 mm, 5 μm column, C-18 Security Guard Cartridge 4.0×3.0 mm and Security Guard Cartridge Holder were used for the analysis. (A) 100% ACN and (B) distilled water containing 0.25% formic acid (Wako, Japan) were used as mobile phase. The gradient conditions of the solvents are shown in Table 3 below. The solvents were flowed at a rate of 1 ml per minute at a column temperature of 30° C., 10 μl was injected, and absorbance was measured at 370 nm.

TABLE 3

| Time (min) | A % | B % |
|---|---|---|
| 0 | 25 | 75 |
| 3 | 25 | 75 |
| 33 | 70 | 30 |
| 40 | 90 | 10 |
| 45 | 90 | 10 |
| 45.5 | 25 | 75 |
| 50 | 25 | 75 |

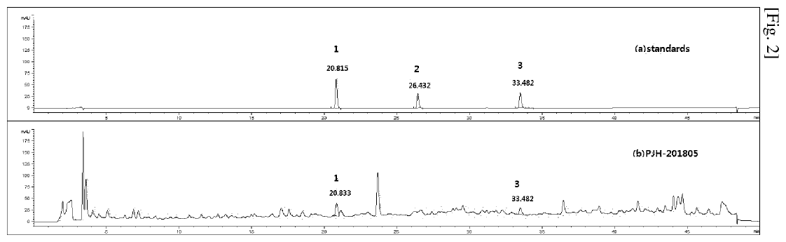

The standard product was extracted by ultrasonic for 10 minutes using 100% methanol at a concentration of 1 mg/l ml. Each standard product was aliquoted and stored in a −20° C. freezer, and used in dilution for analysis. The extract sample was placed in a 50 ml volumetric flask to a concentration of about 500 mg/50 ml, and sonicated for 20 minutes by adding 40 ml of 100% methanol. When cooled to room temperature, the flask was added with 100% methanol to the indicated line and mixed well. Then, the extract sample was filtered through 0.45 μm PTFE filter and analyzed. HPLC analysis results of the major components of the hops extract are shown in Table 4 below and FIG. 2 (1: isoxanthohumol (IXN), 2: 8-prenylnaringenin (8-PN), 3: xanthohumol).

TABLE 4

| Code | IXN % | Xanthohumol % | 8-PN % |
|---|---|---|---|
| PJH-20180210 | 0.18 | 0.21 | |
| PJH-20180412 | 0.15 | 0.17 | |
| PJH-20180131 | 0.09 | 0.18 | |
| PJH-20180210 | 0.13 | 0.17 | |
| Hopstainer | 0.14 | 1.81 | 0.014 |

### Experimental Example 3: HPLC Analysis of the Combination of Red Clover Extract and Hops Extract

In order to analyze the active ingredient contents for each of the 3:1 and 2:1 combinations of the red clover extract and the hops extract, one representative active ingredient for each substance was selected, and a single analytic method was developed and used. Biochanin A for the red clover extract and xanthohumol for the hops extract were selected and analyzed. Luna C18 4.5×250 mm, 5 μm column, C-18 Security Guard Cartridge 4.0×3.0 mm and Security Guard Cartridge Holder were used for the analysis. (A) 100% ACN and (B) distilled water containing 0.25% formic acid were used as mobile phase. The gradient conditions of the solvents are shown in Table 5 below. The solvents were flowed at a rate of 1 ml per minute at a column temperature of 30° C., 10 μl was injected, and absorbance was measured at 320 nm.

TABLE 5

| Time (min) | A % | B % |
|---|---|---|
| 0 | 35 | 65 |
| 5 | 35 | 65 |
| 34 | 60 | 40 |
| 40 | 90 | 10 |
| 45 | 90 | 10 |
| 47 | 35 | 65 |
| 55 | 35 | 65 |

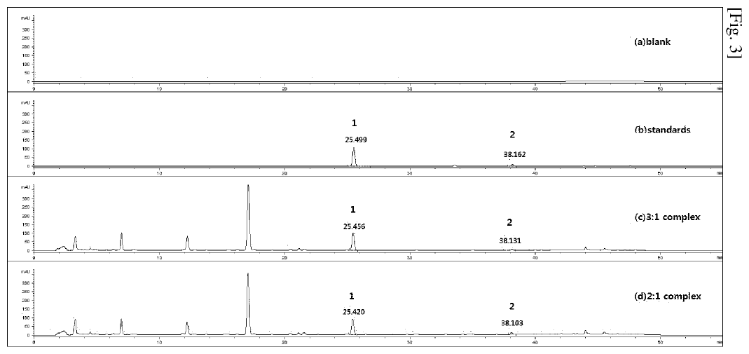

The standard products were each extracted by ultrasonic for 10 minutes using 100% methanol at a concentration of 1 mg/l ml. Each standard product was aliquoted and stored in a −20° C. freezer, and used in dilution for analysis. The extract sample was placed in a 50 ml volumetric flask to a concentration of about 600 mg/50 ml, and sonicated for 20 minutes by adding 40 ml of 100% methanol. When cooled to room temperature, the flask was added with 100% methanol to the indicated line and mixed well. Then, the extract sample was filtered through 0.45 μm PTFE filter and analyzed. HPLC analysis results of (1) biochanin A and (2) xanthohumol in the combination of the red clover extract and the hops extract are shown in FIG. 3.

### Example 1: Measurement of Estrogen Receptor Binding Capacity (ERα-, ERβ-Binding)

Estrogen receptor binding capacity of the combination was measured using PolarScreen™ ER Alpha, Beta Competitor Assay, Green Kit (Life Technologies Inc.). Fluormone™ ES2 Green was used as competitor to measure fluorescence polarization. When Fluormone™ ES2 Green binds to the estrogen receptor (ER), the molecular weight increases, thereby the fluorescence polarization increases. When other substances compete with Fluormone™ ES2 Green, their binding capacity to ER weakens and dissociates, and accordingly the molecular weight is reduced, thereby the fluorescence polarization decreases. This principle can be used to interpret interactions between biological substances. ER was treated with the samples at each concentration ranging from 0.13 to 100 μg/ml, reacted at room temperature for 2 hours, and fluorescence polarization was measured at a wavelength of 485/535 nm using a microplate reader. The observed results were expressed as the percentage inhibition rate relative to the maximum fluorescence polarization value (Max) and compared. E2 (17β-estradiol, Sigma Chem. Co., USA) was used as a positive control.

Fluorescence values according to the sample concentrations were measured, and the concentration that renders half the maximum fluorescence value was determined as IC₅₀ value and is shown in Table 6 below.

TABLE 6

| IC50 (μg/ml) | Estradiol | 3:1 | 2:1 | Red clover | Hops |
|---|---|---|---|---|---|
| ERα | 0.001948 | 5.96 | 8.64 | 5.86 | 30.87 |
| ERβ | 0.001874 | 1.66 | 1.85 | 1.27 | 27.35 |

The IC₅₀ values of the 3:1 and 2:1 combination samples were 5.96 and 8.64 μg/ml for ERβ, respectively, and 1.66 and 1.85 μg/ml for ERβ, respectively. Hence, it was found that both samples have a relatively stronger binding capacity to ERβ.

In addition, in order to confirm the synergistic effect of the combination sample, the inhibitory effect expected from the combination was calculated using the Colby equation (Colby, S. R. Calculation of the synergistic and antagonistic response of herbicide combinations. Weeds 1967, 15, 20-22). The Colby equation is as follows.

Colby expected value=A+B−(A×B/100)

A=Observed value of efficacy of active ingredient A at the same concentration as used in the combination

B=Observed value of efficacy of active ingredient B at the same concentration as used in the combination

As a result, as shown in Table 7 below, a synergistic effect greater than an additive effect was confirmed in both the 2:1 and 3:1 combination samples, and it was observed that the combination sample showed stronger binding capacity than the single sample especially at low concentration.

TABLE 7

| | | 3:1 | | | 2:1 | | |
|---|---|---|---|---|---|---|---|
| ER type | Sample | 3:1 | Red clover | Hops | 2:1 | Red clover | Hops |
| ERα | Conc. (μg/ml) | 11.11 | 8.33 | 2.78 | 11.11 | 7.41 | 3.70 |
| | Inhibition(%) | 81.51 | 80.83 | 0.26 | 80.64 | 78.07 | 0.26 |
| | Colby estimate | 80.88 | | | 78.12 | | |
| | Conc. (μg/ml) | 3.70 | 2.78 | 0.93 | 3.70 | 2.47 | 1.23 |
| | Inhibition(%) | 64.11 | 54.90 | 0.26 | 57.28 | 52.15 | 0.26 |
| | Colby estimate | 55.01 | | | 52.27 | | |
| ERβ | Conc. (μg/ml) | 3.70 | 2.78 | 0.93 | 3.70 | 2.47 | 1.23 |
| | Inhibition(%) | 105.03 | 87.23 | 3.61 | 99.82 | 83.49 | 4.63 |
| | Colby estimate | 87.69 | | | 84.25 | | |
| | Conc. (μg/ml) | 1.23 | 0.92 | 0.31 | 1.23 | 0.82 | 0.41 |
| | Inhibition(%) | 54.41 | 46.07 | 1.60 | 51.98 | 39.83 | 1.77 |

| | | |
|---|---|---|
| Colby estimate | 46.94 | 40.89 |

### Experimental Example 4: MCF-7 and HUVEC Cell Culture

MCF-7 cells (human breast cancer cells) were obtained from the Korea Cell Line Bank and used. RPMI (Gibco, USA) medium containing 10% FBS (Gibco, USA) and 1% penicillin-streptomycin (Gibco, USA) was used as cell culture medium, and cells were cultured in 5% CO₂, 37° C. incubator. The medium was replaced every two days, and cells were sub-cultured every 4-5 days. HUVECs (human umbilical vein endothelial cells) were obtained from LONZA and used. M200 (Gibco, USA) containing LSGS kit (Gibco, USA) was used as cell culture medium, and only cells at passage 3-5 were used for the experiment.

### Example 2: MCF-7 Cells Proliferation Assay

Estrogen-free medium, which was prepared by diluting phenol red-free RPMI (Gibco, USA) with 5% dextran-coated charcoal-stripped FBS (TCB, USA) and 1% penicillin-streptomycin, was used for the experiment. After reacting cells in the estrogen-free medium for 3 days, they were seeded in a 96-well plate at a concentration of 1.5×10⁴ cells/well and incubated in 5% CO₂, 37° C. incubator for 24 hours, and then were treated with the sample so that the final DMSO concentration was 0.1%. E2 was used as the positive control, and ICI 182,780 (Sigma Chem. Co., USA) was used as the negative control. After 72 hours of sample treatment, MTS analysis was performed using CellTiter 96®Aqueous One Solution (Promega, Germany) reagent. Each well was treated with 20 μl of MTS reagent, reacted in 5% CO₂, 37° C. incubator for 3 hours, and the absorbance was measured at 490 nm using a microplate reader.

The results of measuring cell proliferation effect using MCF-7 cells to evaluate estrogen activity are shown in Tables 8 and 9 below. Table 8 shows that cell proliferation was observed in the treatment with the 3:1 combination, the 2:1 combination and the red clover, compared to the control group. In particular, when compared to the red clover and the hops alone treatment, a greater proliferative effect was observed in the treatment with the 3:1 combination or the 2:1 combination, which indicates that the two extracts show a synergistic effect. In order to ascertain whether each sample acts on the estrogen receptor to show a cell proliferation effect, the sample was treated together with the estrogen receptor inhibit ICI 182,780, and the results are shown in Table 9. Table 9 shows that the cell proliferation effect was suppressed when the sample was treated together with the estrogen receptor inhibitor. Hence, it was confirmed that the MCF-7 cell proliferation effect shown in Table 8 is due to each sample activating the estrogen receptor.

TABLE 8

| | Conc. | Proliferation | | Conc. | Proliferation |
|---|---|---|---|---|---|
| Sample | (μg/ml) | (%) | Sample | (μg/ml) | (%) |
| Control | 0 | 100.00 ± 2.26 | E₂ | 1 nM | 135.78 ± 2.83* |
| 3:1 | 0.1 | 109.56 ± 4.80* | 2:1 | 0.1 | 104.00 ± 6.21 |
| | 0.25 | 113.14 ± 1.91* | | 0.25 | 110.01 ± 3.20* |
| | 0.5 | 123.70 ± 4.82* | | 0.5 | 117.20 ± 3.02* |
| red clover | 0.1 | 100.40 ± 5.90 | hops | 0.1 | 96.17 ± 1.76 |
| | 0.25 | 101.42 ± 5.19 | | 0.25 | 94.52 ± 4.24 |
| | 0.5 | 114.94 ± 7.07* | | 0.5 | 103.85 ± 5.35 |

*p < 0.05 vs Control

TABLE 9

| without ICI 182,780 | | | with ICI 182,780 | | |
|---|---|---|---|---|---|
| | Conc. | Proliferation | | Conc. | Proliferation |
| Sample | (μg/ml) | (%) | Sample | (μg/ml) | (%) |
| Control | 0 | 100.00 ± 3.04 | Control | 0 | 86.55 ± 1.95 |
| E₂ | 1 nM | 143.39 ± 10.46 | E₂ | 1 nM | 92.27 ± 2.04 |
| 3:1 | 2 | 137.62 ± 9.12 | 3:1 | 2 | 96.27 ± 2.04 |
| 2:1 | 2 | 133.41 ± 8.28 | 2:1 | 2 | 89.74 ± 2.39 |
| red | 2 | 131.69 ± 8.61 | red clover | 2 | 88.07 ± 3.26 |
| clover | | | | | |
| hops | 2 | 123.10 ± 10.00 | hops | 2 | 85.81 ± 2.50 |

### Example 3: Measurement of Production of Nitric Oxide (NO) and Endothelin-1 (ET-1)

In order to evaluate the blood circulation improvement effect of the combination, the production of nitric oxide (NO), which is one of the vascular relaxation-related biomarkers, and endothelin-1, which is one of the vascular constriction-related biomarkers, was measured using HUVECs. 3×10⁴ cells were seeded in a 96-well plate, cultured for 24 hours and then maintained in a medium under serum starvation condition for 4 hours. In a phenol red-free M200 medium containing 5% charcoal-dextran treated FBS and 1% penicillin-streptomycin, cells were treated with the sample. The final DMSO concentration was 0.5%. Culture supernatant was obtained after 24 hours reaction. NO levels (Tables 10 and 11) and ET-1 levels (Tables 12, 13 and 14) secreted into the culture supernatant were measured using Nitric Oxide analysis kit (R&D systems, USA) and Endothelin-1 Quantikine ELISA kit (R&D systems, USA).

As a result of measuring the NO level, as shown in Table 10 below, the NO level in the culture supernatant was significantly increased when treated with the 3:1 combination or the 2:1 combination, compared to the control group. In order to confirm a synergistic effect, 100 μg/ml of the 3:1 combination, and 75 μg/ml of the red clover and 25 μg/ml of the hops, which are contained in the combination, were each treated, and the efficacies were compared. As shown in Table 11 below, the 3:1 combination group showed a significantly higher increase effect than the single sample group.

TABLE 10

| Sample | Conc. (μg/ml) | NO (%) |
|---|---|---|
| Control | 0 | 100.0 ± 3.7 |
| 3:1 | 10 | 103.7 ± 5.9 |
| | 50 | 126.7 ± 1.0* |
| | 100 | 153.1 ± 2.0* |
| 2:1 | 10 | 106.9 ± 4.5 |
| | 50 | 148.3 ± 10.8* |
| | 100 | 182.7 ± 2.5* |

*p < 0.05 vs Control

TABLE 11

| Sample | Conc. (μg/ml) | NO (%) |
|---|---|---|
| Control | 0 | 100.0 ± 1.5 |
| 3:1 | 100 | 150.0 ± 2.8 |
| red clover | 75 | 124.9 ± 4.3* |
| hops | 25 | 117.6 ± 3.1* |

*p < 0.05 VS 3:1

In addition, as a result of measuring the endothelin-1 level, as shown in Table 12 below, the endothelin-1 level in the culture supernatant was significantly decreased when treated with the 3:1 combination or the 2:1 combination, compared to the control group.

TABLE 12

| Sample | Conc. (μg/ml) | Endothelin-1 (%) |
|---|---|---|
| Control | 0 | 100.0 ± 5.0 |
| 3:1 | 10 | 104.3 ± 8.4 |
| | 50 | 98.0 ± 3.5 |
| | 100 | 82.2 ± 2.1* |
| 2:1 | 10 | 100.8 ± 6.6 |
| | 50 | 92.2 ± 3.5* |
| | 100 | 79.3 ± 4.3* |

*p < 0.05 vs Control

In order to confirm a synergistic effect, 100 μg/ml of the 3:1 combination, and 75 μg/ml of the red clover and 25 μg/ml of the hops, which are contained in the combination, were each treated, and the efficacies were compared. As shown in Table 13 below, the 3:1 combination group showed a significantly greater reduction effect than the single sample group.

TABLE 13

| Sample | Conc. (μg/ml) | Endothelin-1 (%) |
|---|---|---|
| Control | 0 | 100.00 ± 2.9 |
| 3:1 | 100 | 83.1 ± 1.6 |
| red clover | 75 | 95.4 ± 2.4* |
| hops | 25 | 100.2 ± 2.3* |

*p < 0.05 vs 3:1

In order to confirm a synergistic effect of the combination sample, the inhibitory effect expected from the combination was calculated using the Colby equation. As shown in Table 14 below, the Colby estimated value for the inhibition rate of endothelin-1 production was 4.44%, while the actually observed value was 16.9%. Hence, it was confirmed that the 3:1 combination sample has a synergistic effect on the inhibition of endothelin-1 production, compared to when the red clover sample and the hops sample are applied individually.

TABLE 14

| | 3:1 | red clover | hops |
|---|---|---|---|
| Conc. (μg/ml) | 100 | 75 | 25 |
| Inhibition (%) | 16.9 | 4.6 | −0.2 |

| | |
|---|---|
| Colby estimate | 4.4 |

### Example 4: Measurement of eNOS Expression Level

In order to confirm the mechanism of increasing the NO level of the combination, the expression level of endothelial nitric oxide synthase (eNOS) protein, was measured using HUVECs. Cells were seeded in a 24-well plate at a concentration of 1.5×10⁵ cells/well, cultured for 24 hours and then maintained in a medium under serum-free condition for 4 hours. In a phenol red-free M200 medium containing 5% charcoal-dextran treated FBS and 1% penicillin-streptomycin, cells were treated with the sample. The final DMSO concentration was 0.5%. Cell lysate was obtained after 24 hours reaction, and the expression level of eNOS protein was measured using human eNOS DuoSet ELISA kit (R&D systems, USA).

As a result of measuring the expression level of eNOS protein, as shown in Table 15 below, it was confirmed that the eNOS protein expression was significantly increased when treated with the 3:1 combination or the 2:1 combination, compared to the control group. In order to confirm a synergistic effect, 100 μg/ml of the 3:1 combination, and 75 μg/ml of the red clover and 25 μg/ml of the hops, which are contained in the combination, were each treated, and the efficacies were compared. As shown in Table 16 below, the 3:1 combination group showed a significantly higher increase effect than the single sample group.

TABLE 15

| Sample | Conc. (μg/ml) | eNOS (%) |
|---|---|---|
| Control | 0 | 100.0 ± 5.1 |
| 3:1 | 10 | 106.8 ± 6.0 |
| | 50 | 112.2 ± 3.6* |
| | 100 | 150.5 ± 13.8* |
| 2:1 | 10 | 122.5 ± 24.8 |
| | 50 | 126.1 ± 8.4* |
| | 100 | 156.7 ± 35.8* |

*p < 0.05 vs Control

TABLE 16

| Sample | Conc. (μg/ml) | eNOS (%) |
|---|---|---|
| Control | 0 | 100.00 ± 3.6 |
| 3:1 | 100 | 148.4 ± 9.5 |
| red clover | 75 | 123.3 ± 11.1* |
| hops | 25 | 115.0 ± 10.6* |

*p < 0.05 vs 3:1

### Experimental Example 5: Animal Test Evaluation Method

In this experiment, the effect of the combination of red clover and hops extract for increasing physiological activity on menopausal disorders was evaluated using bi-lateral ovarioectomized rat model (OVX). Specifically, 250 and 500 mg/kg/day of the 2:1 (w/w) and 3:1 (w/w) combinations of red clover extract and hops extract were dissolved or dispersed in sterile distilled water, and orally administered to rats after removal of the ovaries (4 days after surgery) once a day at a dose of 10 ml/kg for 56 days (8 weeks). The effects of these combinations on body weight, tail skin temperature (TST), serum lipid profile, bone metabolism, antioxidant, markers of vasorelaxation and uterus endometrium were evaluated.

### (1) Experimental Animals

Female virgin Sprague-Dawley rats (11 weeks old, OrientBio, Seungam, Korea) were obtained and used after 1 week of adaptation. This experiment was conducted under the examination and approval of Naturetech Animal Experiment Ethics Committee (Approval No. UIK21702).

### (2) Experiment Method

In order to induce menopausal disorders due to estrogen deflection, all experimental animals except the sham operated control group were randomly selected and conducted the bi-lateral ovariectomy. The sham operated control group was conducted same surgery procedure excepted eliminating the ovaries. For all experimental animals including the sham operated control group, clinical symptoms and body weight were measured 3 or 4 days after surgery, and animals with no abnormalities were selected and grouped by randomization according to their weight.

Each experimental group was given the following name:

Sham: Sham operated control group

OVX: Bi-lateral ovarioectomized group

2:1 (500): Group administered with the 2:1 (w/w) combination sample of red clover extract and hops extract at a dose of 500 mg/kg/day

2:1 (250): Group administered with the 2:1 (w/w) combination sample of red clover extract and hops extract at a dose of 250 mg/kg/day

3:1 (500): Group administered with the 3:1 (w/w) combination sample of red clover extract and hops extract at a dose of 500 mg/kg/day

3:1 (250): Group administered with the 3:1 (w/w) combination sample of red clover extract and hops extract at a dose of 250 mg/kg/day

Information related to each experimental group is shown in Table 17 below.

TABLE 17

| Group | Sample | Dose (mg/kg) | No. animals |
|---|---|---|---|
| Sham | Vehicle | 0 | 8 |
| OVX | Vehicle | 10 | 8 |
| OVX + 2:1(500) | Red clover:Hops (2:1) | 500 | 9 |
| OVX + 2:1(250) | | 250 | 9 |
| OVX + 3:1(500) | Red clover:Hops (3:1) | 500 | 9 |
| OVX + 3:1(250) | | 250 | 9 |

### Example 5: Results of Change in Body Weight and Body Weight Gain after 8 Weeks of Administration of the Combination of Red Clover Extract and Hops Extract

The 2:1 combination and the 3:1 combination of red clover extract and hops extract were orally administered to OVX rats for 8 weeks, and body weight change and body weight gain change were observed. As a result, significant inhibition of body weight increase was observed in the 500 mg/kg dose group of both combinations. For body weight gain, it was observed that body weight gain was significantly inhibited in the 500 mg/kg dose group of the 2:1 combination, and in both the 250 and 500 mg/kg dose groups of the 3:1 combination (Table 18).

TABLE 18

| | Body weight (g) | | Body weight gain (g) | |
|---|---|---|---|---|
| Group | (mean ± SD) | P value | (mean ± SD) | P value |
| Sham | 322.3 ± 22.2 | 0.0002 | 52.2 ± 16.8 | 0.0001 |
| ovx | 391.6 ± 33.2 | — | 115.4 ± 30.2 | — |
| OVX + 2:1 (500) | 357.6 ± 29.3 | 0.0402 | 81.8 ± 24.2 | 0.0223 |
| OVX + 2:1 (250) | 384.2 ± 24.0 | 0.6030 | 108.5 ± 16.2 | 0.5578 |
| OVX + 3:1 (500) | 340.8 ± 36.0 | 0.0088 | 65.3 ± 29.8 | 0.0037 |
| OVX + 3:1 (250) | 365.4 ± 24.5 | 0.0813 | 89.5 ± 19.4 | 0.0499 |

### Example 6: Measurement of Feed Efficiency Change

The average body weight increase (weight gain) and the average feed intake of rats during the administration period were calculated and converted to FER (food efficacy ratio) which is shown in Table 19 below. According to Table 19, the 2:1 combination significantly reduced FER in the 500 mg/kg dose group, and the 3:1 combination significantly reduced FER in both the 250 and 500 mg/kg dose groups.

TABLE 19

| | Body weight gain (g/day) | | Feed intake(g/day) | | FER(%) | |
|---|---|---|---|---|---|---|
| Group | (mean ± SD) | P value | (mean ± SD) | P value | (mean ± SD) | P value |
| Sham | 0.9 ± 0.3 | 0.0001 | 14.9 ± 1.3 | 0.0962 | 6.2 ± 1.8 | 0.0001 |
| OVX | 2.1 ± 0.5 | — | 16.3 ± 1.7 | — | 12.6 ± 2.8 | — |
| OVX + 2:1 (500) | 1.5 ± 0.4 | 0.0223 | 14.8 ± 1.0 | 0.0413 | 9.8 ± 2.7 | 0.0589 |
| OVX + 2:1 (250) | 1.9 ± 0.3 | 0.5578 | 17.2 ± 2.0 | 0.3102 | 11.3 ± 1.0 | 0.2416 |
| OVX + 3:1 (500) | 1.2 ± 0.5 | 0.0037 | 14.7 ± 1.4 | 0.0596 | 7.8 ± 3.0 | 0.0040 |
| OVX + 3:1 (250) | 1.6 ± 0.3 | 0.0499 | 15.3 ± 1.3 | 0.1938 | 10.4 ± 1.7 | 0.0701 |

### Example 7: Measurement of Tail Skin Temperature (TST)

TST (tail skin temperature) in the animal model relates to the effect of improving hot flashes, which is one of the main symptoms of menopause. After 8 weeks of administration, the TST of the all animals was measured by an infrared thermometer (Bioseb, USA), and the increase in tail skin temperature was significantly suppressed in the 500 mg/kg dose group of both combinations (Table 20).

TABLE 20

TST (° C.)

| Group | (mean ± SD) | P value |
|---|---|---|
| Sham | 26.9 ± 0.8 | 0.1299 |
| OVX | 27.6 ± 0.8 | — |
| OVX + 2:1 (500) | 26.2 ± 0.5 | 0.0008 |
| OVX + 2:1 (250) | 27.0 ± 1.1 | 0.2079 |
| OVX + 3:1 (500) | 26.6 ± 0.9 | 0.0353 |
| OVX + 3:1 (250) | 27.1 ± 1.4 | 0.3617 |

### Example 8: Change in Weight of Organs

At the end of experiment, all animals after fasting over 16 hours were autopsied, blood samples were collected and organs were extracted, and the weight of the wet organs was measured (Table 21). A significant decrease in the fasted body weight was observed in the 500 mg/kg dose group of both combinations, the same as observed during the experiment period.

A significant decrease in uterine weight was observed in the OVX group compared to the sham operated control group, and this decrease did not change significantly even when the two combinations were administered.

On the other hand, as a result of measuring the wet weight of the abdominal fat mass, a significant decrease was observed in the 500 mg/kg dose group of both combinations, the same as observed in the body weight change. Hence, it can be assumed that the weight loss effect by the 2:1 combination and the 3:1 combination was due to suppression of visceral fat accumulation.

TABLE 21

| | Fasted BW | | Uterus | | Visceral fat | |
|---|---|---|---|---|---|---|
| Group | (mean ± SD) | P value | (mean ± SD) | P value | (mean ± SD) | P value |
| Sham | 308.70 ± 23.76 | 0.000 | 0.76 ± 0.37 | 0.002 | 4.06 ± 0.94 | 0.000 |
| OVX | 377.97 ± 31.36 | — | 0.15 ± 0.13 | — | 7.07 ± 1.51 | — |
| OVX + 2:1 (500) | 345.21 ± 30.19 | 0.046 | 0.22 ± 0.20 | 0.415 | 4.95 ± 2.07 | 0.030 |
| OVX + 2:1 (250) | 361.55 ± 35.36 | 0.333 | 0.18 ± 0.17 | 0.667 | 8.25 ± 1.56 | 0.138 |
| OVX + 3:1 (500) | 327.76 ± 33.01 | 0.006 | 0.27 ± 0.22 | 0.214 | 4.30 ± 1.08 | 0.001 |
| OVX + 3:1 (250) | 351.66 ± 23.71 | 0.071 | 0.12 ± 0.01 | 0.441 | 6.37 ± 2.76 | 0.534 |

### Example 9: Blood Lipid Change

As a result of measuring blood lipids in the experimental animals, a significant decrease in low density lipoprotein (LDL) was observed in the 500 mg/kg dose group of the 2:1 combination of red clover extract and hops extract, and in the 250 and 500 mg/kg dose groups of the 3:1 combination. In the case of total cholesterol (TC), the 2:1 combination administered group showed a decrease pattern, although not significant, and the 250 and 500 mg/kg dose groups of the 3:1 combination showed a significant decrease (Table 22).

TABLE 22

| | Total-Chol (mg/dL) | | LDL-C(mg/dL) | |
|---|---|---|---|---|
| Group | (mean ± SD) | P value | (mean ± SD) | P value |
| Sham | 93.7 ± 11.0 | 0.314 | 8.0 ± 1.4 | 0.011 |
| OVX | 101.5 ± 14.3 | — | 11.6 ± 2.5 | — |
| OVX + 2:1 (500) | 87.6 ± 12.8 | 0.091 | 8.2 ± 1.6 | 0.013 |
| OVX + 2:1 (250) | 96.3 ± 10.1 | 0.460 | 9.9 ± 2.2 | 0.206 |
| OVX + 3:1 (500) | 75.7 ± 16.5 | 0.013 | 7.6 ± 2.1 | 0.009 |
| OVX + 3:1 (250) | 79.1 ± 11.2 | 0.009 | 7.9 ± 1.8 | 0.011 |

### Example 10: Changes in Osteocalcin, ALP and CTX-1 in Blood

It is known that the level of osteocalcin, ALP and CTX-1 in blood is an indicator reflecting bone metabolism, of which osteocalcin and ALP are indicators reflecting bone formation, and CTX-1 is an indicator reflecting bone resorption. Postmenopausal women show a significant decrease in bone density as well as significantly higher levels of the above three indicators. Hence, reduction of these indicators means improved bone metabolism by the combination of red clover extract and hops extract. In this experiment, the effect of the combination of red clover extract and hops extract on blood osteocalcin and ALP content was observed.

Significant increase in blood osteocalcin and blood ALP contents was observed in the OVX control group compared to the sham operated control group, whereas a significant decrease in blood osteocalcin and blood ALP content was observed in the groups administered with the 2:1 combination and 3:1 combination of red clover extract and hops extract (Table 23).

TABLE 23

| | Serum ALP | | Osteocalcin (pg/ml) | |
|---|---|---|---|---|
| Group | (mean ± SD) | P value | (mean ± SD) | P value |
| Sham | 54.2 ± 14.9 | 0.004 | 1286.5 ± 318.1 | 0.092 |
| OVX | 80.7 ± 6.7 | — | 1714.5 ± 463.6 | — |
| OVX + 2:1 (500) | 68.3 ± 16.1 | 0.122 | 1262.3 ± 238.8 | 0.045 |
| OVX + 2:1 (250) | 68.0 ± 8.1 | 0.021 | 1789.3 ± 352.5 | 0.747 |
| OVX + 3:1 (500) | 58.7 ± 16.9 | 0.016 | 1632.4 ± 227.8 | 0.686 |
| OVX + 3:1 (250) | 69.4 ± 4.3 | 0.011 | 1541.4 ± 256.7 | 0.413 |

In addition, the effect of the combination of red clover extract and hops extract on blood CTX-1 content was observed. As a result of analyzing the blood concentration of CTX-1 in the experimental animals, a significant decrease in blood CTX-1 was observed in the 500 mg/kg dose group of both the 2:1 combination and 3:1 combination of red clover extract and hops extract (Table 24).

TABLE 24

CTX-1 (pg/dL)

| Group | (mean ± SD) | P value |
|---|---|---|
| Sham | 11.1 ± 1.8 | 0.008 |
| OVX | 14.3 ± 2.1 | — |
| OVX + 2:1 (500) | 11.7 ± 1.3 | 0.035 |
| OVX + 3:1 (500) | 10.4 ± 2.9 | 0.032 |

### Example 11: Blood Endothlin-1 and NO Changes

In this example, the effect of the combination of red clover extract and hops extract on blood endothelin-1 and NO contents was observed.

As a result of analyzing the blood concentrations of endothelin-1 and NO in the experimental animals, a significant decrease in blood endothelin-1 and a significant increase in blood NO were observed in the 250 and 500 mg/kg dose groups of the 3:1 combination of red clover extract and hops extract (Table 25).

TABLE 25

| | Endothlin-1 | | NO | |
|---|---|---|---|---|
| Group | (mean ± SD) | P value | (mean ± SD) | P value |
| Sham | 100.0 ± 14.0 | 0.0010 | 100.0± 21.5 | 0.0051 |
| OVX | 151.2 ± 28.1 | — | 69.4 ± 9.9 | — |
| OVX + 3:1 (250) | 115.3 ± 30.0 | 0.0176 | 77.4 ± 3.8 | 0.0707 |
| OVX + 3:1 (500) | 94.3 ± 14.7 | 0.0005 | 92.0 ± 9.9 | 0.0012 |

### Example 12: Blood SOD and MDA Changes

In this example, the effect of the combination of red clover extract and hops extract on blood SOD and MDA contents was observed.

As a result of analyzing the blood concentrations of SOD and MDA in the experimental animals, a significant increase in blood SOD and a significant decrease in blood MDA were observed in the 250 and 500 mg/kg dose groups of the 3:1 combination of red clover extract and hops extract (Table 26).

TABLE 26

| | SOD (U/ml) | | MDA (uM/ml) | |
|---|---|---|---|---|
| Group | (mean ± SD) | P value | (mean ± SD) | P value |
| Sham | 42.62 ± 12.87 | 0.0046 | 10.24 ± 3.45 | 0.0006 |
| OVX | 17.42 ± 14.27 | — | 19.22 ± 3.86 | — |
| OVX + 3:1 (250) | 46.71 ± 11.37 | 0.0011 | 13.74 ± 3.50 | 0.0165 |
| OVX + 3:1 (500) | 48.70 ± 14.95 | 0.0017 | 11.54 ± 4.61 | 0.0055 |

### Example 13: Change in Endometrial Height

In this example, in order to observe the impact of the combination of red clover extract and hops extract on the endometrial height, the extracted uterine tissue after autopsy was stained to measure the height of the endometrium.

The height of endometrial epithelial cells was significantly decreased in the OVX control group compared to the sham operated control group, and the 250 and 500 mg/kg dose groups of the 3:1 combination of red clover extract and hops extract did not show a significant difference from the OVX control group (Table 27).

TABLE 27

Height of epithelial cells (μm)

| Group | (mean ± SD) | P value |
|---|---|---|
| Sham | 27.71 ± 5.36 | 0.0000 |
| OVX | 14.34 ± 2.78 | — |
| OVX + 3:1 (250) | 14.30 ± 2.63 | 0.1755 |
| OVX + 3:1 (500) | 14.97 ± 2.59 | 0.3478 |

The foregoing descriptions of the present invention are intended for illustration, and it should be understood by those skilled in the art that the present invention may be easily modified in other specific embodiments without changing the technical concept or essential features of the present invention. Therefore, it should be understood that the examples described above are exemplary and not limiting in all respects. For example, each component described as a single type may be implemented in a separate manner, and similarly, components described as being separated may be implemented in a combined form.

The scope of the present invention is represented by the following claims rather than the above detailed description, and all changes or modifications derived from the meaning and scope of the claims and their equivalents should be construed as being included in the scope of the present invention.

1. A composition comprising as an active ingredient a combination of a red clover extract and a hops extract.

2. The composition according to claim 1, wherein the combination comprises the red clover extract and the hops extract in a weight ratio of 10:1 to 1:5.

3. The composition according to claim 2, wherein the combination comprises the red clover extract and the hops extract in a weight ratio of 3:1 to 2:1.

4. (canceled)

5. The composition according to claim 1, wherein the red clover extract is extracted from a red clover leaf.

6. The composition according to claim 1, wherein the hops extract is extracted from hops flowers, hops buds, or both.

7. The composition according to claim 1, wherein the red clover extract and the hops extract are each red clover extract powder and hops extract powder.

8. The composition according to claim 1, which comprises a therapeutically effective amount of the red clover extract and the hops extract shows to have a synergistic effect compared to when the red clover extract and the hops extract are administered individually.

9. The composition according to claim 1, which comprises a therapeutically effective amount of the red clover extract and the hops extract to have one or more of the following activities:
(1) Estrogen receptor binding
(2) Improvement of estrogen activity
(3) Increase of blood nitric oxide (NO) level
(4) Increase of blood eNOS protein expression level
(5) Decrease of blood endothelin-1 level
(6) Suppression of visceral fat accumulation
(7) Decrease of blood lipid
(8) Decrease of blood osteocalcin, ALP and CTX-1 contents
(9) Increase of blood SOD and decrease of blood MDA
(10) Suppression of rat tail skin temperature (TST) increase

10. A food composition comprising the composition according to claim 1.

11. A pharmaceutical composition comprising the composition according to claim 1.

12. The pharmaceutical composition according to claim 11, wherein the pharmaceutical composition is for oral administration, intraperitoneal administration, intravenous administration, intramuscular administration, subcutaneous administration, intradermal administration, intranasal administration, pulmonary administration or rectal administration.

13. The pharmaceutical composition according to claim 12, wherein the pharmaceutical composition is formulated as tablets, pills, powders, granules, capsules or drinks for oral administration.

14. A method for preparing a composition, comprising the following steps:
(a) pulverizing a red clover leaf and then extracting it in alcohol to prepare a red clover extract;
(b) extracting hops flowers and hops buds in alcohol to prepare a hops extract; and
(c) mixing the red clover extract and the hops extract.

15. The method for preparing a composition according to claim 14, wherein the steps (a) and (b) may further include additional steps of filtering, concentrating and drying the extract after alcohol extraction.

16. (canceled)

17. A method of improving, preventing or treating women's menopausal disorders in a subject, comprising:
administering to the subject an effective amount of a combination of a red clover extract and a hops extract.

18. The method according to claim 17, wherein the menopausal disorders represent one or more menopausal symptoms selected from the group consisting of blood circulation disorders, weight gain, hot flashes, osteoporosis, blood cholesterol increase, sweating, insomnia, nervousness, depression, dizziness, lack of concentration, joint pain, headache, palpitations, vagina dryness, fatigue, excitement, sleep disorders, memory loss, memory disorders, forgetfulness, cold sweats, pounding, frequent urination, anxiety and atherosclerosis.
